(19) United States
(12) Patent Application Publication
(10) Pub. No.: US 2015/0122733 A1
(43) Pub. Date: May 7, 2015

(54) Method for the treatment of cancer

(71) Applicant: Marv Enterprises, LLC, El Paso, TX (US)

(72) Inventor: Mitchell S. Felder, El Paso, TX (US)

(21) Appl. No.: 14/378,681

(22) PCT Filed: May 21, 2013
(86) PCT No.: PCT/US2013/041923

§ 371 Date: Aug. 14, 2014

Related U.S. Application Data
(60) Provisional application No. 61/650,316, filed on May 22, 2012.

(51) Int. Cl. C07K 16/30; A61K 47/48; A61K 39/395; A61M 1/34; A61M 1/16

(52) U.S. Cl. CPC C07K 16/30; A61M 1/342; A61M 1/16; A61K 39/39558; A61K 47/48284; A61M 2202/0413
USPC 210/638

(57) ABSTRACT

The present invention relates to the treatment of cancers. Specifically, the invention pertains to a method for the extracorporeal treatment of one or more body fluids in two stages, characterized by removing a body fluid from a living body diseased with a type of cancer, passing the body fluid through a first stage, applying an anti-angiogenesis, anti-tumorigenesis, anti-metastasis, or chemotherapeutic treatment to at least one antigen in the body fluid. More specifically, the treatment comprises creating an antibody-antigen moiety during passage through the first stage, passing the treated body fluid through a second stage, removing the antibody-antigen moiety from the body fluid during passage through the second stage, and returning the purified body fluid to the body. The invention is further characterized by targeting an antigen in the body fluid, with an antibody to allow and facilitate removal thereof in the second stage.

### CROSS-REFERENCE TO RELATED APPLICATIONS

This application claims benefit under 35 U.S.C. §119(e) of U.S. Patent Application No. 61/650,316, filed May 22, 2012, which is hereby incorporated herein by reference in its entirety.

### BACKGROUND OF THE INVENTION

In the United States cancer is the second leading cause of death. Cancer has surpassed heart disease is the number one cause of death in patients younger than 85 years of age. In the United States each year 1.3 million cases of cancer are diagnosed and more than 570,000 people die each year from cancer. The highest mortality rates involving lung, colorectal, breast and prostate cancers. More people presently die from cancer in the United States each year than were killed in a all of the wars in which the United States participated during the twentieth century. The average age at the time of diagnosis for cancer is 67 years, and about three quarters of all cancers are diagnosed after the age of 55. Further, cancer is the second leading cause of death in children up to the age of fourteen. In children leukemia is the most common cause of death from cancer. There are over 8 million Americans alive today who have had some form of cancer. Approximately one in three Americans will develop some form of cancer within their lifetime.

The spreading of cancer within a body by metastasis appears to be facilitated by the formation of new blood vessels supplying the cancer cells with nutrients. Angiogenesis is the physiological process of the growth of new blood vessels from pre-existing vessels. These new blood vessels supply nutrients to the cancer cells and facilitate growth of malignant tumors and the spread of cancer cells to other parts of the body.

Certain molecular organic compounds are implicated as causing or allowing angiogenesis which in turn allows the metastasis of various cancer cells and growth of cancerous tumors.

### SUMMARY OF THE INVENTION

In general terms, the present invention relates to the treatment of cancers, hereinafter abbreviated as “CA”. Specifically, the invention pertains to a method for the extracorporeal treatment of one or more Body Fluids in two stages characterized by removing a Body Fluid from a living body diseased with a type of CA, passing the Body Fluid through a first stage; applying an anti-angiogenesis, anti-tumorigenesis, anti-metastasis, or chemotherapeutic treatment to at least one antigen in the Body Fluid.

More specifically, the treatment comprises creating an antibody-antigen moiety during passage thereof through said first stage; passing the treated Body Fluid through a second stage; removing antibody-antigen moiety from the Body Fluid during passage through the second stage, and returning the purified Body Fluid to the body.

The invention is further characterized by targeting an antigen in the Body Fluid, with an antibody to allow and facilitate removal thereof in the second stage. The targeted antigens would include one, or a combination of:
1. Antigens involved in causing or facilitating Angiogenesis (CA Ant. Ang.):
   A. VEGF: Vascular endothelial growth factor;
   B. VEGFR: Vascular endothelial growth factor receptor tyrosine kinase inhibitor;
   C. NRP-1: Neurolipin-1;
   D. Ang1: Angiopoietin 1;
   E. Tie2: Tyrosine kinase/CD 202B (Cluster of differentiation 202B);
   F. PDGF-BB: platelet derived growth factor;
   G. Endoglin: CD105;
   H. TGF-beta: Transforming growth factor beta;
   I. FGF: Fibroblast growth factor;
   J. HGF: Hepatocyte growth factor/scatter factor;
   K. MCP-1: Monocyte chemotactic protein-1;
   L. Integrins: heterodimers with alpha and beta subunits;
   M. VE-cadherin: Vascular endothelial-cadherin; CD144 (Cluster of Differentiation 144), Cadherin 5, type 2;
   N. PECAM: Platelet Endothelial Cell Adhesion Molecule/CD 31 (Cluster of differentiation molecule);
   O. Matrix metalioproteinase: 2,3,7 and 9;
   P. PAI-1 : Plasminogen Activator Inhibitor-1;
   Q. CXC Chemokines;
   R. Id1/Id3: Inhibitors of differentiation;
2. Antigens involved in causing or facilitating Tumorigenesis; (CA Ant. T.):
   A. IGF-1: Insulin-like growth factor 1;
   B. IGF-2: Insulin-like growth factor 2;
   C. FGF: Fibroblast growth factor;
   D. NGF: Nerve growth factor;
   E. PDGF: Platelet derived growth factor;
   F. Tumor growth factor: alpha and beta;
3. Antigens involved in a Signal Transducer; (CA Ant. ST):
   A. Sonic hedgehog homolog: (SHH);
   B. Indian hedgehog homolog: (IHH);
   C. COX2: Cyclooxygenase-2;
4. Antigens which are unique to specific CAs (examples); (CA Ant. Sp.):
   A. MTA1: Metastasis associated protein 1 (breast cancer);
   B. AGR2: Anterior gradient 2 (adenocarcinomas of the pancreas, esophagus, prostate, lung cancer);
   C. Tau protein (breast cancer);
   D. GL12 (melanoma metastasis);
   E. Integrin alpha3beta1 (breast cancer);
   F. CCL25 (Ovarian cancer);
   G. Kif18A (Breast cancer);
   H. MMP9 (Nasopharyngeal carcinoma);
   I. Type 1 gamma phosphatidylinositol phosphate kinase (Breast cancer);
   J. Ubc9 (Breast cancer);
5. Antigens which decrease chemotherapeutic efficacy of treatments (example); (CA Ant. Chem.):
   A. Interleukin-6.

Specifically, the method is further characterized by removing Body Fluid from a person to produce the extracorporeal bodily fluid; imposing a treatment acting on an antigen of CA in the Body Fluid, filtering or otherwise removing the treatment from the Body Fluid, and returning the Body Fluid to the patient after removing substantially all of the treatment in the second stage.

The method of the present invention comprises treating at least one component of a patient's Body Fluid extracorporeally with a designer antibody containing an albumin-moiety which will create an albumin-antibody-CA antigen moiety allowing for the efficacious dialysis of the resultant albumin-antibody-CA antigen compound (the targeted CA antigen being respectively, one or a combination of antigen(s) from: CA Ant. Ang., CA Ant. T., CA Ant. ST, CA Ant. Sp., CA Ant. Chem.).

More specifically, the method is characterized by removing Body Fluid from a person to produce the extracorporeal bodily fluid; directing a first antibody against the targeted CA antigen (CA Ant. Ang., CA Ant. T., CA Ant. ST, CA Ant. Sp., CA Ant. Chem.) in the first stage of extra-corporeal treatment in the Body Fluid; in the second stage directing a second antibody conjugated with albumin and/or a protein against the targeted CA antigen thereby forming an albumin-antibody-CA antigen compound; removing at least a substantial portion of the albumin-antibody-CA antigen compound from the Body Fluid by dialysis, other filtering, or other means; and returning the Body Fluid to the patient.

Also, the method is characterized by testing the blood and/or CSF to determine the efficacy of treatment before returning the Body Fluid to the patient.

### BRIEF DESCRIPTION OF THE DRAWINGS

FIG. 1 is a partial cross sectional view of a cylinder and tubing used to deliver a treatment to a bodily fluid.

FIG. 2 is a partial cross sectional view showing additional detail of the cylinder and tubing of FIG. 1.

### DETAILED DESCRIPTION OF THE INVENTION

In the first stage of treatment, a selected Body Fluid is removed using a standard catheter and/or lumbar puncture. In the second stage, the Body Fluid is treated with antibodies against the targeted CA antigen (CA Ant. Ang., CA Ant. T., CA Ant. ST, CA Ant. Sp., CA Ant. Chem.).

The method of the present invention comprises treating at least one component of a patient's Body Fluid extracorporeally with a designer antibody containing an albumin-moiety to create an albumin-antibody-CA antigen moiety allowing for the efficacious dialysis, filtering or other means of removal of the resultant albumin-antibody-CA antigen compound (the targeted CA antigen being respectively, one or a combination of antigen(s) from: CA Ant. Ang., CA Ant. T., CA Ant. ST, CA Ant. Sp., CA Ant. Chem.).

The albumin-antibody will be directed towards facilitating removal of the targeted CA antigen(s): CA Ant. Ang., CA Ant. T., CA Ant. ST, CA Ant. Sp., CA Ant. Chem. After the removal of the CA antigen(s), the cleansed Body Fluid will be returned to the patient. The frequency of treatment, and the specifically targeted CA antigen(s) to be removed would depend upon the underlying symptomatology and pathology of the patient, and would be determined by the patient's physician. The article used in performing the method includes two-stages. The first stage includes a treatment chamber for addition of an antibody with an attached albumin moiety, which is added to the Body Fluid. A second stage receives the treated blood and/or CSF and includes a unit for removing the treatment.

The method includes providing a dialysis or other filtering machine with the a first stage and a second stage, and sequentially passing the extracorporeal Body Fluid through the first and second stages. The Body Fluid is removed from the patient using standard procedure. The first stage applies a treatment using an antibody which was has attached to it an albumin moiety (or alternatively, a moiety which allows for the efficacious dialysis or removal by other techniques of the antibody-albumin-CA antigen), for the treatment of the Body Fluid. The second stage substantially removes the treatment. The purified Body Fluid (Body Fluid with removed targeted CA antigen: CA Ant. Ang., CA Ant. T., CA Ant. ST, CA Ant. Sp., CA Ant. Chem.—is then tested for the efficacy of removal of the CA antigen and returned to the patient.

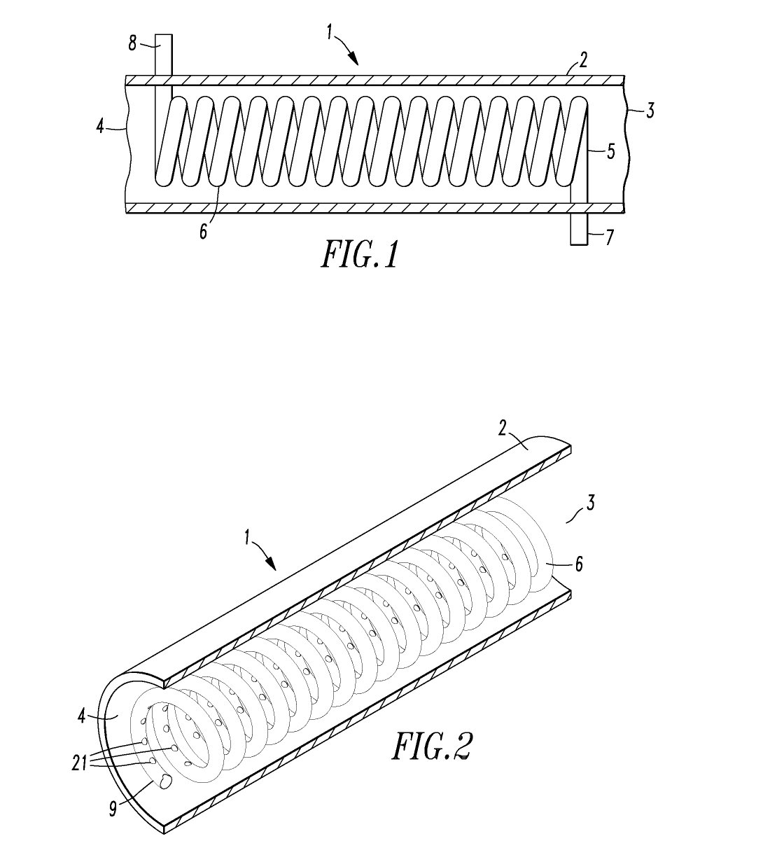

The device of the invention includes a first stage and a second stage. The first stage applies a treatment of an antibody with an attached albumin moiety targeting the CA antigen(s) specifically exacerbating the pathologic condition. The second stage includes substantial removal of the treatment from the extracorporeal Body Fluid bodily fluid. As shown in FIG. 1, the first stage can include an exterior wall to define a treatment chamber 5. The treatment conveniently can be applied in the treatment chamber 5. Residence times of the Body Fluid can be altered by changing the dimensions of the treatment chamber, or by using a dialysis vacuum pump. With reference to FIG. 1, Body Fluid enters the inlet 3, passes through the treatment chamber 5, and exits the outlet 4. In embodiments, the treatment of an antibody with an attached albumin moiety targeting the CA antigen(s) can be applied from a delivery tube 6 located within the treatment chamber 5. An inferior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the blood and/or CSF. As shown, the delivery tube 6 comprises a helical coil.

With reference to FIG. 2, when the treatment includes the administration of a designer antibody, the delivery tube 6 can be hollow and the interior wall 9 can define a plurality of holes 21. The designer antibodies can be pumped through the delivery tube 6 in order to effect a desired concentration of designer anti bodies in the Body Fluid. The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic. The antibody with attached albumin moiety, targeting the CA antigen(s) can be delivered in a concurrent or counter-current mode with reference to the Body Fluid. In counter-current mode, the Body Fluid enters the treatment chamber 5 at the inlet 3. The designer antibody can enter through a first lead 8 near the outlet 4 of the treatment chamber 5. The blood and/or CSF then passes to the outlet 4 and the designer antibodies pass to the second lead 7 near the inlet 3. The removal module of the second stage substantially removes the designer antibodies-CA antigen molecular compound from the Body Fluid.

The second stage can include a filter, such as a dialysis machine, which is known to one skilled in the art. The second stage can include a molecular filter. For example, molecular adsorbents recirculating system (MARS), which may be compatible and/or synergistic with dialysis equipment. MARS technology can be used to remove small to average sized molecules from the Body Fluid. Artificial liver filtration presently uses this technique.

The method can include a plurality of steps for removing the targeted CA antigen(s). A first step can include directing a first antibody against the targeted antigen. A second step can include a second antibody. The second antibody can be conjugated with albumin, or alternatively another moiety which allows for efficacious dialysis or filtering of the antibody-CA antigen from the Body Fluid. The second antibody or antibody-albumen complex combines with the first antibody forming an antibody-antibody-moiety complex. A third step is then used to remove the complex from the blood and/or CSF. This removal is enabled by using dialysis and/or MARS. The purified Body Fluid is then returned to the patient.

In practice, a portion of the purified Body Fluid can be tested to ensure a sufficient portion of the targeted CA antigen(s) have been successfully removed from the Body Fluid. Testing can determine the length of treatment and evaluate the efficacy of the sequential dialysis methodology in removing the targeted CA antigen(s) and suggest the need for further treatment. Body Fluid with an unacceptably large concentrations of complex remaining can then be retreated and refiltered before returning the Body Fluid to the patient.

In embodiments, the second stage to remove the antibody-moiety-targeted CA antigen complex from the Body Fluid can be accomplished by various techniques including, for example, dialysis, filtering based on molecular size, protein binding, solubility, chemical reactivity, and combinations thereof. For example, a filter can include a molecular sieve, such as zeolite, or porous membranes that capture complexes comprising molecules above a certain size. Membranes can comprise polyacrylonitrile, polysulfone, polyamides, cellulose, cellulose acetate, polyacrylates, polymethylmethacrylates, and combinations thereof. Increasing the flow rate or diasylate flow rate can increase the rate of removal of the antibody with attached albumin moiety targeting the CA antigen(s) such as CA Ant. Ang., CA Ant. T., CA Ant. ST, CA Ant. Sp., CA Ant. Chem.

Further techniques can include continuous renal replacement therapy (CRRT) which can remove large quantities of filterable molecules from the extracorporeal Body Fluid. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis. The sieving coefficient (SC) is the ratio of the molecular concentration in the filtrate to the incoming CSF. A SC close to zero implies that the moiety-antibody-targeted antigen complex will not pass through the filter. A filtration rate of 50 ml per minute is generally satisfactory. Other methods of increasing the removability of the antibody-targeted antigen moiety include the use of temporary acidification of the Body Fluid extracorporeally using organic acids to compete with protein binding sites.

Numerous modifications and variations of the present invention are possible. It is, therefore, to be understood that within the scope of the following claims, the invention may be practiced otherwise than as specifically described. While this invention has been described with respect to certain preferred embodiments, different variations, modifications, and additions to the invention will become evident to persons of ordinary skill in the art. All such modifications, variations, and additions are intended to be encompassed within the scope of this patent, which is limited only by the claims appended hereto.

All documents, books, manuals, papers, patents, published patent applications, guides, abstracts and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for the extracorporeal treatment of a body fluid in two stages characterized by:
passing the body fluid through a device having a first stage;
applying a treatment to at least one CA antigen in the body fluid to create an antibody-CA antigen moiety during passage thereof through said first stage;
passing the treated body fluid through a second stage; and
removing the treatment (antibody-CA antigen moiety) from the body fluid during passage through said second stage.

2. The method of claim 1 characterized by targeting a CA antigen in said body fluid, such as antigens involved in angiogenesis (CA Ant. Ang.).

3. The method of claim 1 characterized by targeting a CA antigen in said body fluid, such as antigens involved in tumorigenesis (CA Ant. T.).

4. The method of claim 1 characterized by targeting a CA antigen in said body fluid, such as antigens involved as a signal transducer (CA Ant. ST).

5. The method of claim 1 characterized by targeting a CA antigen in said body fluid, such as antigens which are unique to specific carcinomas (CA Ant. Sp.).

6. The method of claim 1 characterized by targeting a CA antigen in said body fluid, such as antigens which decrease chemotherapeutic efficacy (CA Ant. Chem.):

7. The method of claim 1 characterized by targeting an antigen in said body fluid selected from the group consisting of CA Ant. Ang., CA Ant. T., CA Ant. ST, CA Ant. Sp., and CA Ant. Chem.

8. The method of claim 1 characterized by:
directing a first antibody against the targeted CA antigen in the first stage; and
directing a second antibody conjugated with albumin and/or a protein against the targeted antigen, thereby forming an albumin-antibody-CA antigen compound in the second stage; and
removing at least a portion of the albumin-antibody-CA antigen compound from the body fluid.

9. The method of claim 1 further characterized by:
removing blood and/or CSF from the patient to produce the extracorporeal body fluid; and
returning the body fluid to the patient after substantially removing the treatment in the second stage.

6. The method of claim 5 characterized by testing the body fluid for the efficacy of treatment before returning the body fluid to the patient.
